(19) United States
(12) Patent Application Publication
(10) Pub. No.: US 2010/0331649 A1
(43) Pub. Date: Dec. 30, 2010

(54) INTERACTIVE ANALYSIS METHOD FOR BIOSIGNALS

(76) Inventor: Chang-An Chou, Taipei (TW)

Correspondence Address: Chang-An Chou, 3F, No. 100, Sec. 3. Mingsheng E. Rd., Taipei, 105, TW

(21) Appl. No.: 12/865,858

(22) PCT Filed: Feb. 5, 2009
(86) PCT No.: PCT/CN09/00132

§ 371 Date: Aug. 2, 2010

(30) Foreign Application Priority Data
Feb. 5, 2008 (CN) 200810080711.9

(51) Int. Cl. A61B 5/021; A61B 5/00; A61B 5/0402; A61B 5/087; A61B 5/0476; A61B 5/11; A61B 5/0488; A61B 5/145; G06F 3/048; G06F 21/00

(52) U.S. Cl. 600/364; 600/300; 600/509; 600/538; 600/544; 600/595; 600/546; 600/485; 600/365; 715/764; 726/17

(57) ABSTRACT

An interactive analysis method for biosignals is disclosed. The method includes steps of providing at least a biosignal acquisition device, a user utilizing the biosignal acquisition device to acquire at least one kind of biosignals therefrom, the user uploading the acquired biosignals to a service platform through a network, wherein the service platform provides at least a set of processing means for said kind of biosignals with at least an algorithm included therein, the user selecting at least a desired processing means from the set of processing means, the service platform performing the selected processing means on the uploaded biosignals for producing an analysis result, the user deciding a handling flow for the analysis result, the user deciding a handling flow for the uploaded biosignals, and the service platform performing the handling flows decided by the user. Therefore, through the biosignal interactive analysis method, not only the users can customize their own operation procedure, but the physiological examination and diagnosis also can be performed at any desired time and location once the biosignal acquisition device is connected to the service platform via network.

### FIELD OF THE INVENTION

The present invention is related to an interactive analysis method for biosignals, and more particularly to a biosignal analysis method which provide various kinds of processing means and feedbacks real-time analysis result via network, and also provides user the opportunity to select and decide the contents of analysis.

### BACKGROUND OF THE INVENTION

Owing to increased health consciousness, modern people demand for more understandings to their own physical conditions, so the development of home medical equipment is getting increased.

Another reason for developing home medical equipment is chronic disease. Because chronic disease needs long-term treatment and monitor and thus consumes a lot of medical resources, the home medical equipment provides one way to save medical resources.

Most home medical equipments are focused on those examinations require only simple values, such as, blood pressure and blood sugar, so that the users can estimate their own physical condition by easily interpreting if the shown value falls within the specific range or not, for example, if blood pressure/blood sugar is too high or too low. Therefore, this kind of diagnosis, that only requires examination and does not involve in complex analysis, is common at home and hospital.

However, since more and more medical resources has consumed by chronic disease, as well as the patients nowadays have developed higher demands for understanding their own physical conditions, homecare diagnosis is led to be developed toward a more professional way and no longer be limited to show simple values, where the portable EKG device is one of the examples, such as, HCG-801 (a handheld electrocardiometer produced by Omron), and SCS-H10/H20 (an electrocardiogram recording device manufactured by TOSHIBA). The portable EKG device is usually designed with built-in analysis software to provide EKG analysis result for the user, so the patient who has cardiovascular disease can monitor his condition at home. As compared to the blood pressure meter, the portable EKG device is obviously a more sophisticate home medical equipment for monitoring cardiovascular disease.

Nevertheless, the disadvantage for such home medical equipment with built-in simple analysis software is that the analysis provided thereby are fixed, so that the understanding of the patient for his/her own physical condition is also limited thereby. Besides, it is also difficult to change the analysis content even the requirement of patient has already changed.

Accordingly, there are two kinds of homecare systems have been developed to solve this problem. One example is the Sentry Telehealth Monitor developed by Honeywell Hommed, the other is a remote real-time physiological diagnostic system developed by Televital. Both in common are for patient to perform physiological diagnostics, which was usually executed in hospital, at home, so both systems involve participation of physician. No matter the physiological data is transmitted to medical personnel via telephone line (Hommed) or the patient can have a real-time communication with medical personnel on Internet (Televital), both require the medical personnel to analyze and interpret the physiological signals derived and then send the analyzed result back to the patient verbally.

Although this kind of methodology allows the patient to communicate with the medical personnel at home, the limitation is the participation of medical personnel. For example, as using the Hommed's system, the diagnosis of patient is basically guided by medical personnel, so the user just performs the examination organized by the physician and then uploads the data. Therefore, the user's physical condition is long-term monitored by the medical personnel and the patient is informed passively.

For the remote real-time physiological diagnostic system of Televital, although it provides a professional physiological diagnosis to the patient, the medical personnel must involve the diagnosis. Thus, the diagnosis might not be performed if the medical personnel are not available or busy. This makes the homecare diagnosis not available at anytime by user's will.

Generally speaking, one of the main purposes for homecare diagnosis is to let the patients to perform the diagnosis by themselves. This not only allows the patient to keep control of his/her physical condition, but also lets the medical resources being used efficiently, so a win-win situation can be achieved. Therefore, a feasible homecare diagnostic solution with professional diagnostic report and user-friendly interface, and not limited by time or location, is one of the main objectives for homecare diagnosis nowadays. Besides, if the users can select the diagnostic contents by themselves based on their own condition, the acceptance of homecare diagnosis might be further increased.

The object of the present invention is to provide an interactive analysis method for biosignals where the analysis contents can be decided by the user.

Another object of the present invention is to provide an interactive analysis method for biosignals, which offers various selectable processing means for the user by remote service platform, so the diagnostic contents are no more limited by the built-in software of homecare device, and the user can perform a more professional examination at anytime or any location.

Further object of the present invention is to provide an interactive analysis method for biosignals, where the analysis, interpretation and transmission destination of the physiological signals are decided by the user.

### SUMMARY OF THE INVENTION

According to one aspect of the present invention, an interactive analysis method for biosignals is provided. The method includes steps of: providing at least a biosignal acquisition device, a user utilizing the biosignal acquisition device to acquire at least one kind of biosignals therefrom, the user uploading the acquired biosignals to a service platform through a network, wherein the service platform provides at least a set of processing means for said kind of biosignals with at least an algorithm included therein, the user selecting a desired processing means from the set of processing means, the service platform performing the selected processing means on the uploaded biosignals for producing an analysis result, the user selecting a handling flow for the analysis result, the user selecting a handling flow for the uploaded biosignals, and the service platform performing the handling flows selected by the user.

In another aspect of the present invention, an interactive real-time analysis method for biosignals is disclosed. In this case, the method includes steps of providing a service platform, providing at least a biosignal acquisition device, a user connecting the biosignal acquisition device to the service platform via a network, the user utilizing the biosignal acquisition device to acquire at least one kind of biosignals therefrom, and the biosignal acquisition device transmitting the biosignals to the service platform in real time, wherein the service platform provides at least a set of processing means for said kind of biosignals with at least an algorithm included therein. Moreover, before, during or after the biosignal acquisition, the user can decide a handling flow for said acquired biosignals on the service platform, and the service platform performs the handling flow decided by the user. Furthermore, before, during or after the biosignal acquisition, the user also can select at least a processing means related to said kind of biosignals from the set of processing means on the service platform and the service platform performs the selected processing means on the uploaded biosignals for producing an analysis result, and then, the user can decide at least a handling flow for the analysis result and the service platform performs the handling flow decided by the user.

Preferably, the biosignals acquired by the biosignal acquisition device are transmitted to a local device first and then further transmitted to the service platform via a wired or wireless network interface of the local device Moreover, the local device will provides an user interface for the user to control functions provided by the service platform, wherein the user interface can be a web-based user interface, an application-based user interface, or a mixed user interface. Here, the local device is a computer, a PDA, a mobile phone, or a device with networking capability. Alternatively, in a preferred embodiment, the biosignal acquisition device also can be integrated with the local device to be one entity, so as to further simplify the system and also the operation procedure.

Preferably, the acquired biosignals can be one or more selected from a group consisting of: ECG, airflow, snore, respiratory effort, EEG, oxygen saturation, limb movement, EMG, EOG, blood pressure, heart rate, blood sugar, and physiological ultrasonics, without limitation.

Preferably, the handling flows include a medical personnel enters the service platform to access the biosignals and/or the analysis result stored by the user, and in this case, the service platform may play a communication medium between the user and the medical personnel, and the medical personnel also can access user's data as authorized by the user. And, the handling flows also include transmitting the analysis result and/or the biosignals to a medical service provider, wherein the service platform further includes a list of medical personnel who participate in the interactive physiological analysis, so that the user can select the medical service provider from the list or the service platform will randomly select or allocate medical personnel for the user.

Through the interactive analysis method for biosignals of the present invention, the user not only can select the type of biosignal acquisition, but also can freely organize the handling flows of the uploaded biosignals, such as, processing means, medical personnel and the route of the uploaded data, so that the user can customize his/her own operation procedure without limitation. Further, since the examination can be performed at home, the user can actively obtain the physical condition thereof and decide the schedule to meet a doctor or to receive the opinion from professionals. And, owing to the property of network and the processing means provided by the service platform, the physiological examination can be performed at any desired time and location, so that the user can easily access, handle and manage his/her own physiological data without limitation. Therefore, in addition to saving the traveling time to hospital, since many diseases can be better controlled and monitored by this manner, the medical resources also can be utilized more effectively.

### BRIEF DESCRIPTION OF THE DRAWINGS

A more detailed understanding of the invention may be had from the following description of a preferred embodiment, given by way of example, and to be understood in conjunction with the accompanying drawings, wherein:

FIG. 1 is a schematic view showing the architecture of the interactive analysis system of the present invention;

FIG. 2 is a flow chart showing the interactive analysis method for biosignals in a preferred embodiment according to the present invention; and

FIG. 3 a flow chart showing the interactive analysis method for biosignals in another preferred embodiment according to the present invention.

### DETAILED DESCRIPTION OF THE PREFERRED EMBODIMENTS

In view of the drawbacks in the prior arts, the present invention provides a service platform equipped with various kinds of biosignal processing means, so that even the physiological examinations performed at home also can be calculated and analyzed by algorithm through uploading to the service platform, thereby the participation necessity of medical personnel can be reduced, so as to achieve a biosignal diagnosis without time and location limitations and also provide user an additional choice other than seeking for doctor's help.

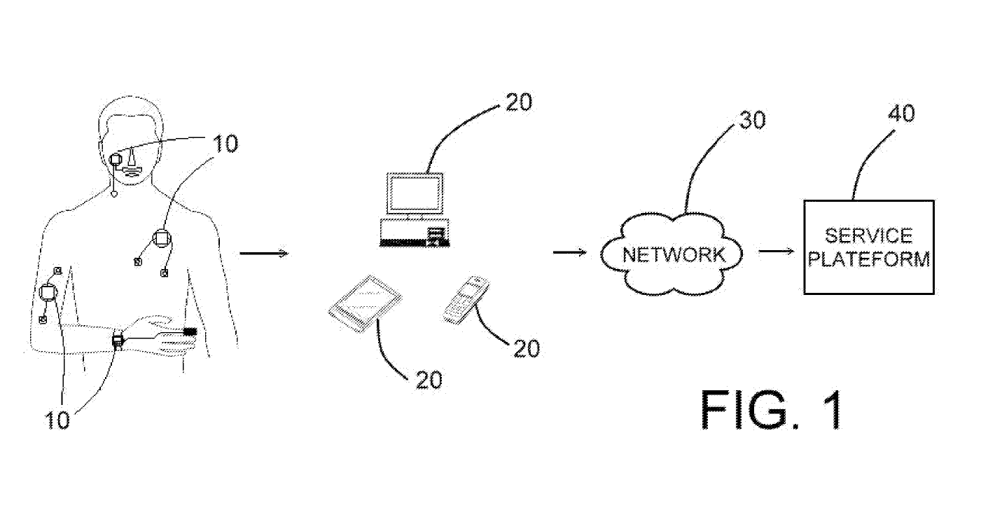

Please refer to FIG. 1 which is a schematic view showing the architecture of an interactive analysis system for biosignals according to the present invention. As shown, the interactive analysis system for biosignals includes at least a biosignal acquisition device 10, a local device 20, a network 30, and a platform 40, wherein the biosignal acquisition device 10 and the local device 20 are located at the user end and the service platform 40 is located at the remote end, and the communication between the biosignal acquisition device 10/local device 20 and the service platform 40 is achieved by the network 30.

The biosignal acquisition device 10 is used for acquiring biosignals from the user. According to the present invention, the biosignal acquisition device 10 is intended to be used by user at home, so that the design thereof focuses on simple installation, easy operation and light weight, thereby reducing the operation difficulty and increasing user's acceptance. Here, there is no limitation to the function of the biosignal acquisition device 10. It can be used to acquire any type of biosiganls capable of being acquired at home, for example, but not limited, ECG, EEG, EMG, EOG, respiration (airflow and respiratory effort), snore, oxygen saturation, limb movement, blood pressure, heart rate, blood sugar, and physiological ultrasonic. Therefore, the user can decide which examination or which kind of examination combination he/she wants to execute without limitation.

After biosignal acquisition, the biosignal acquisition device 10 is then connected to the network 30 via the local device 20 to upload the biosignals to the service platform 40. Accordingly, the local device 20 may include a network interface for connecting to the network 30. Here, the network interface can be wired or wireless network interface, such as, LAN or IEEE 802.11x. Further, the transmission between the biosignal acquisition device 10 and the local device 20 also can be performed to wired or wireless transmission, such as, USB connection or Bluetooth connection. Besides, the local device 20 can be, but not limited, a computer, a PDA, a mobile hone, or a device with networking and displaying functions.

Furthermore, according to the present invention, the service platform 40 is equipped with processing means for various kinds of biosignals, wherein the processing means may include analysis algorithms and signal processing algorithms, and the setting options of each algorithm, such as, parameters and operation conditions, also can be modified by the user.

When receiving uploaded biosignals, according to the type thereof, the service platform 40 will provide a set of processing means, for example, as receiving EKG biosignals, a set of EKG processing means will be formed. In each set of processing means, many algorithms related to that type of biosignals will be included, for example, a set of EKG processing means may include, but not limited, HRV analysis algorithm, heat rate analysis algorithm, waveform analysis algorithm, and multi-channel analysis algorithm; a set of SPO2 processing means may include, but not limited, sleep apnea analysis algorithm, respiratory monitoring analysis algorithm, and HRV analysis algorithm; and a set of EEG processing means may include, but not limited, epilepsy analysis algorithm, melancholia analysis algorithm, and brain wave analysis algorithm.

Moreover, other than the analysis algorithms, the set of processing means also may include signal processing algorithm(s), for example, digital filter processing algorithm, frequency domain processing algorithm, parametric/characteristic processing algorithm, and wavelet processing algorithm, but not limited.

As well-known, one type of biosignals, according to different physiological symptoms, can be analyzed by different kinds of analysis algorithms, so that if suitable analysis algorithms and/or different parameters can be provided according to different symptoms, the interpretation of physiological conditions will be more appropriate. More importantly, when there comes a new analysis algorithm or signal processing algorithm, the user can directly use it on the service platform without upgrading the program in the biosignal acquisition device.

Therefore, as long as network connection is available, the user can obtain the analysis result through selected processing means in the service platform 40 at any time.

Consequently, the main purpose of the present invention is that: the user only needs to accomplish the operation of biosignal acquisition, and the following processing and analysis can be handed over to the service platform after network upload. The user can freely select which kind of biosignals is going to acquire according to the real demands, without being restricted by limited choices.

Plus, in the interactive analysis system for biosignals according to the present invention, it is also important that user can manipulate the entire operation process.

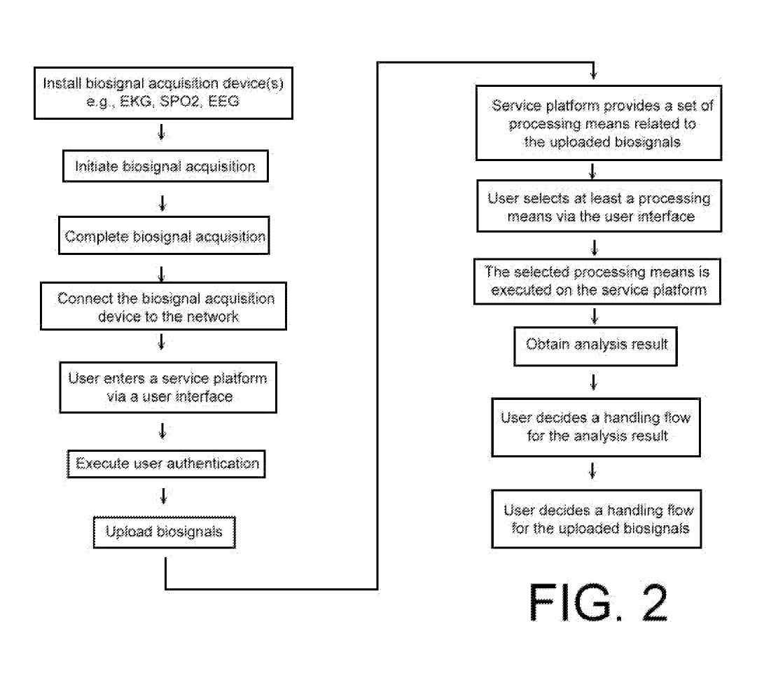

The following is the description of a preferred embodiment according to the present invention by employing single biosignal acquisition device. FIG. 2 shows the operation steps of the interactive analysis system. However, it should be noticed that

the number of the biosignal acquisition device can be implemented to be multiple, and the category thereof also can be more than one.

### Step 1 Installation of Biosignal Acquisition Device

According to the characteristics of different biosignal acquisition devices, the installation positions thereof are also different. For example, oximeter is disposed on the finger tip, EKG electrodes are disposed on the chest, EEG electrodes are disposed on the scalp, snore sensor is disposed on throat or nose, and respiratory sensor is disposed between nose and mouth or on the thorax and/or abdomen. That is, the disposing position will be changed depending on different biosignal acquisition devices.

### Step 2 Initiation of Biosignal Acquisition

After installation, the biosignal acquisition can be initiated. According to a preferred embodiment, the biosignal acquisition device is provided with a start button, so that the user can easily initiate the acquisition by pressing the start button. In another preferred embodiment, the system may further include a control device for controlling the operation of the biosignal acquisition device, such as, start and stop.

### Step 3 Networking, Authentication, Upload

After biosignal acquisition, the biosignal acquisition device is connected to the local device and link to the network via the local device, so as to upload the biosignals to the service platform. At this time, the user can utilize a user interface provided by the local device to enter the service platform. Here, the user interface can be a web-based user interface, an application-based user interface, or a mixed user interface, that is, the user interface can be a webpage as the user enters the service platform, or the user interface can be provided by the software installed in the local device, or the user interface can be provided by the cooperation of the webpage and the software installed in the local device.

Through the guiding of the user interface, the user can easily use the functions provided by the service platform.

Then, first, as entering the service platform, the service platform will perform a user authentication process. Through the connection route established by the biosignal acquisition device, the network and the service platform, the service platform can detect the presence of the biosignal acquisition device and initiate the user authentication process; or the service platform will ask the user to input personal information and security number for authentication, there is no limitation. In the authentication process, the service platform will identify user's identification, which also facilitates to organize or manage the future information inputted by this user. Of course, it is also possible to create different user accounts for the same user to conform to different situations or demands, so that a self-management of biosignals can be achieved.

So far, the user finished the upload process. However, it should be noticed that the sequence of steps 1-3 described above is not specific, and the operation flow can be varied as environment/situation changes. For example:

1. The biosignal acquisition device can be connected to the local device before signal acquisition, so that the acquired biosignals can be transmitted to the local device for storage in real time, and after completing signal acquisition, the local device is then connected to the service platform for data upload.

2. The biosignal acquisition device also can be connected to the service platform via the local device and the network before signal acquisition, that is, the acquired biosignal can be directly stored in the service platform in real time, and under this situation, the user authentication process should be performed in advance so as to authorize the data storage.

Therefore, the sequence of steps of signal acquisition, network connection, authentication and data upload can be varied according to situation differences, and there is no specific restriction thereto.

### Step 4 Selection of Processing Means

After upload, in accordance with the type of biosignals, the service platform will provide a related set of processing means thereto. For example, when the service platform receives uploaded EKG signals from the user, a set of EKG processing means will be provided, including, but not limited, HRV analysis algorithm, heart rate analysis algorithm, waveform analysis algorithm, and multi-channel analysis algorithm, so that the user can select one or more desired processing means therefrom case by case.

That is, according to the present invention, single type of biosignals still can be selected to perform different kinds of processing means for producing analysis/processing results conforming to different demands. Therefore, it is preferable that the service platform is maintained to maximize user's choice, for example, by periodical update and/or upgrade as new processing means is available.

Moreover, it should be noticed that the user can select one or more processing means at a time, or select another after completing the selected one. There is no limitation.

Furthermore, the user also can modify the setting option(s), such as, parameters and operation conditions, of the selected algorithm(s). This is specially convenient for professionals. When medical personnel enters this system for accessing patient's data (as described below), he/she can adjust the parameters and operation conditions of each processing means, for more precisely interpreting the biosignals and thus providing the user a more accurate analysis result and advices.

Here, in a preferred embodiment of the present invention, the medical personnel can enter the service platform through the user interface provided by the local device (no matter the local device is aside the biosignal acquisition device or is another local device remote therefrom), such as, a computer, a PDA, and a mobile phone, so as to perform analysis and setting.

### Step 5 Decision of Handling Flow for Analysis Result

After the selected processing means by the user is executed on the service platform, corresponding analysis/processing result can be obtained, and then, the user can decide a handling flow of the analysis/processing result, for example, to store the result and/or to perform other handling flows provided by the service platform.

For example, the user can decide to transmit the analysis result to the medical personnel who are available on the service platform, for seeking for professional opinions or for asking related questions. Here, the selection of medical personnel can be randomly decided by the service platform, or can be decided by the user from a list by the service platform.

Alternatively, the user can decide not to store the result, for example, when there is no need to review the result, or it found that some unwanted situations occurred in the signal acquisition process, or the acquired signal is not good enough.

Alternatively, the user can decide to compare this analysis result with the already existed analysis result to achieve the purpose of long-term monitoring of physiological conditions.

Moreover, in addition to the selected handling flow for the analysis/processing result, the service platform also will supervise subsequent procedure(s) corresponding thereto. For example, if the user hopes to receive professional opinions from medical personnel, in addition to performing data transmission, the service platform also can be the communication medium between the user and medical personnel, such as, when the opinion returns, the system can inform the user by email or text message; or if the user hopes to contact the medical personnel directly, then under the authorization of the medical personnel, the system can deliver the contact information to the user; or under user's authorization, the system can allow the medical personnel to access user's physiological data and information directly on the service platform; or even, the user and medical personnel can have a direct communication on the service platform. Therefore, through this system, not only the traveling time between patient's home and hospital or clinic, but also the medical resources can be saved.

Here, after medical personnel receives user's physiological data, he/she also can select from the set of processing means provided by the service platform to perform various kinds of analyses at any time or location only if network access is available. For example, when the physician travels to other places for conference or tour, as long as he/she can enter the service platform via network, the communication with the patients will still be maintained without limitation.

Moreover, although the service platform provides the opportunity for the user to interact with medical personnel, the user also can select to bypass data delivery to medical personnel, which is especially useful for user who has stable physical condition and employs this system for routine physiological monitoring. Besides, since the time limitation has been disengaged, user can perform the examination at any free time and no more be restricted by the available time of doctor. Both patients and medical personnel benefit a lot.

More particularly, owing to the feature of network, as long as the user carries the biosignal acquisition device as traveling, the daily monitoring of physical condition still can be achieved through cooperating with a networking local device, such as, laptop, PDA or mobile phone.

### Step 6 Decision of Handling Flow for Biosignals

The user can decide the handling flow of the uploaded biosignals with or without the execution of processing means. The handling flows include, but not limited, show trace, compare with existed biosignals, combined with other biosignals of the same user, storage, transmit to medical personnel, delete etc.

According to one embodiment of the present invention, the user can directly transmit the biosignals to medical personnel with or without executing processing means, or store the biosignals for further access from the medical personnel and inform medical personnel to view the biosignals.

In view of the above, owing to the various selections and functions provided by the service platform, the user can decide the operation procedures freely, and further due to the nature of network, the time and location for the user to access this system are also limitless, so that the user is no more passive but can actively handle the examination and decide what kind of information he/she wants.

Here, it should be noticed that although the above preferred embodiment is described step by step, there is no specific sequence. The timings for executing the selection and decision steps can be varied, especially for real-time operation.

The main operation procedure is the local biosignal acquisition plus the signal upload to remote service platform, and other steps can be executed before, during or after thereof.

For example, the user can enter the service platform and decide the handling flow of the biosignals before, during or after the biosignal acquisition. Alternatively, it is also possible to execute the step of selecting processing means before, during or after the biosignal acquisition, and accordingly, the timing for executing the step of deciding the handling flow of the analysis result is also variable.

In one preferred embodiment, before biosignal acquisition, the user can enter the service platform and decide the processing means and the handling flows for biosignals and analysis result (if applicable), so that as finishing the biosignal acquisition, the operation procedure is also completed. Alternatively, in another preferred embodiment, the user can select and decide the processing means and the handling flows when biosignals are acquiring. Therefore, there is no limitation to the operation sequence of the above steps.

Furthermore, other than the above preferred embodiments employing single biosignal acquisition device, it is also possible to apply multiple biosignal acquisition devices to this system.

According to the concept of the present invention, user can select to buy different types of biosignal acquisition devices depending on personal needs. For example, at the beginning, if only the blood oxygen level is required, then an oximeter should be bought only, and later, as requirements of other examinations arise, the user can further buy other biosignal acquisition devices, such as EKG detector, to assist physiological interpretation. Alternatively, the medical personnel also can provide the user the combination of biosignal acquisition devices which are more suitable for the current physical condition thereof.

As uploading biosignals, if the analysis needs to compare multiple types of biosignals, for example, analysis of sleep physiology might need multiple types of sleep related biosignals, such as, respiration, snore, EKG, oxygen saturation, EMG etc., then also through selection, the user may ask the service platform to execute a synchronization and/or combination operation to multiple biosignals.

Moreover, the service platform also may provide processing means for different combinations of biosignals. When the combination of biosignals uploaded by the user changes, correspondingly, the service platform can provide different choices of processing means for conforming thereto. Therefore, simply through user's decision at each step, the service platform can accordingly provide a custom-made operation procedure for every user, which means, the user plays an active but not passive role in this system.

Of course, the synchronization of multiple biosignals also might be unnecessary, so that the user also can select not to combine multiple biosignals and perform respective processing means thereof.

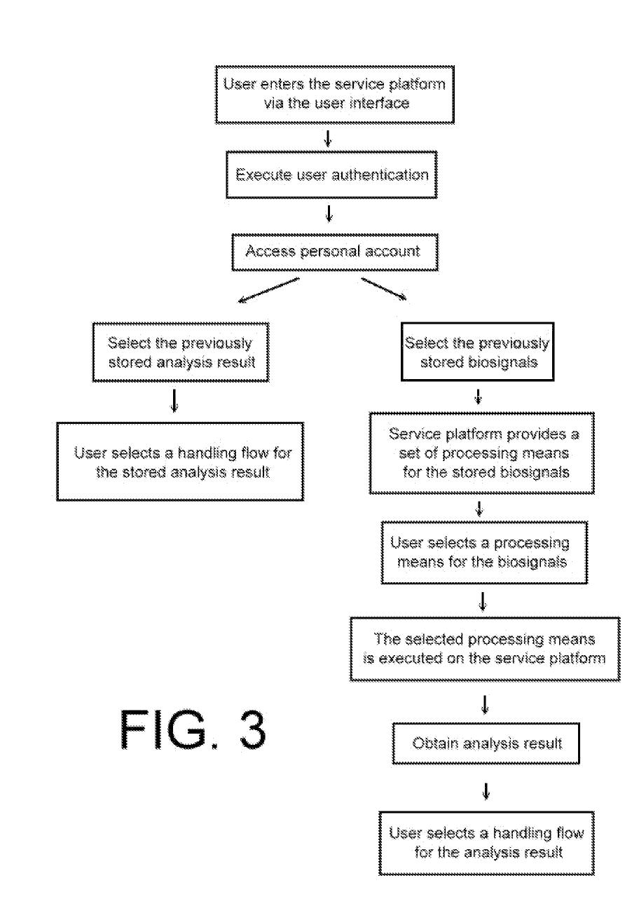

Besides, the service platform also can provide operations other than data upload and analysis. For example, the user can simply enter the service platform to review his/her data stored previously, such as, signal traces and analysis results, as shown in FIG. 3, after the authentication process. At this time, the user can perform all operations provided by the service platform to the stored data, such as, different kinds of processing means, or transmit the analysis result to medical personnel, so that the user can access the uploaded data at any time or location as the network is existed.

Advantageously, the user can utilize the service platform to manage his/her personal physiological information. For example, as the user is periodically tracked owing to chronic disease, such as, heart disease, he/she can use this system to check the effects of diet control and medicine taken, such as, by measuring EKG and/or blood pressure, and under the authorization of user, the medical personnel also can access the tracking result through the service platform, so that even patient and doctor do not have a face to face consultation, the disease still can be monitored and controlled well, and more advantageously, time cost and medical resources both can be saved. Of course, the user also can utilize the service platform to monitor personal physical condition without medical personnel's participation. For example, it can use to examine the sleep physiology or the brain activity of the user.

In the past, except certain items, such as, blood pressure and blood sugar, most kinds of physiological examinations should be executed under physician's supervision, so the patient has to meet the doctor for every examination, which means it is difficult for the patient to monitor the physical condition at home. However, through the interactive system provided by the present invention, this problem can be solved, and it becomes easier to realize one's own physical condition.

In another preferred embodiment, for simplifying operation, the biosignal acquisition device and the local device also can be integrated into one entity, that is, the biosignal acquisition device with network connection and user interface, for example, a mobile phone or PDA with networking and biosignal acquisition functions, could be a possible example. Thus, the implementation of the biosignal acquisition device and the local device can be varied in accordance with different situations without limitation.

Moreover, in addition to select at every step and every time, the user also can configure a preset route of procedure in this system, for example, which kind of processing means and which medical personnel will the uploaded data submitted to, and the system will automatically run the customized route every time the user uploads data so as to omit the selection steps. Of course, the settings can be varied as user's demand changes.

In addition, for ensuring data security, especially as the data in this system is transmitted via the network, it is performed to encrypt all data, for example, biosignals, personal information, information of medical personnel etc.

Besides, since this system can gather data from large amount of users and the data might be constantly updated by users themselves, the whole database will become a great resource for statistics, such as, the arrangement and trend of certain disease in different gender, age, human race and/or residence. And, as more users participate in this system, the statistic result will become more accurate, which provides benefit to medical research. Further, based on the statistic result, the service platform also can provide selection suggestions, for example, which processing means is selected more frequently and which doctor is most popular, so as to guide the user during the step-by-step procedure.

In the aforesaid, through the interactive analysis system for biosignals according to the present invention, the user not only can select the type of biosignal acquisition, but also can freely organize the handling flows of the uploaded biosignals, such as, which processing means, which medical personnel and what kind of route the uploaded data will be submitted to, so that the user can customize his/her own operation procedure without limitation. Further, the user can actively decide the schedule to meet a doctor or to receive the opinion from professionals and no more be passively instructed thereby. And, most importantly, the physiological examination and diagnosis can be performed at any desired time and location once the biosignal acquisition device can be connected to the service platform via network, so that the user can easily access, handle and manage his/her own physiological data as needed. Therefore, in addition to saving the traveling time to hospital, the medical resources also can be utilized more effectively.

What is claimed is:

1. An interactive analysis method for biosignals, comprising steps of:
providing at least a biosignal acquisition device;
a user utilizing the biosignal acquisition device to acquire at least one kind of biosignals therefrom;
the user uploading the acquired biosignals to a service platform through a network, wherein the service platform provides at least a set of processing means for said kind of biosignals with at least an algorithm included therein;
the user selecting at least a desired processing means from the set of processing means;
the service platform performing the selected processing means on the uploaded biosignals for producing an analysis result;
the user deciding a handling flow for the analysis result;
the user deciding a handling flow for the uploaded biosignals; and
the service platform performing the handling flows decided by the user.

2. The method as claimed in claim 1, wherein the algorithm is implemented to be analysis algorithm and/or signal processing algorithm.

3. The method as claimed in claim 2, wherein the signal processing algorithm is related to time domain, frequency domain, parameter/characteristic or wavelet analysis.

4. The method as claimed in claim 2, wherein the parameters and operation conditions of the analysis algorithm and the signal processing algorithm are capable of be modified by the user.

5. The method as claimed in claim 1, wherein the biosignals acquired by the biosignal acquisition device are transmitted to a local device first and then further transmitted to the service platform,

6. The method as claimed in claim 5, wherein the local device has a network interface for connecting to the network.

7. The method as claimed in claim 6, wherein the network interface is a wireless network interface or wired network interface.

8. The method as claimed in claim 5, wherein the local device provides a user interface for the user to control functions provided by the service platform.

9. The method as claimed in claim 8, wherein the user interface is a web-based user interface, an application-based user interface, or a mixed user interface.

10. The method as claimed in claim 5, wherein the local device is a computer, a PDA, a mobile phone, or a device with networking capability.

11. The method as claimed in claim 1, wherein the biosignal acquisition device is integrated with the local device to be one entity.

12. The method as claimed in claim 1, wherein said at least one kind of biosignals is one or more biosignals selected from a group consisting of: ECG, airflow, snore, respiratory effort, EEG, oxygen saturation, limb movement, EMG, EOG, blood pressure, heart rate, blood sugar, and physiological ultrasonics.

13. The method as claimed in claim 1, wherein when the biosignal acquisition device is connecting to the service platform, the service platform performs an authentication to identify the user.

14. The method as claimed in claim 1, wherein the handling flow of the biosignals includes viewing the trace of the biosignals.

15. The method as claimed in claim 1, wherein the handling flow of the biosignals includes combining the acquired biosignaled with other biosignals of the same user.

16. The method as claimed in claim 1, wherein the handling flow of the biosignals includes comparing the acquired biosignals with a second biosignal.

17. The method as claimed in claim 16, wherein the second biosignal is stored by the user in advance.

18. The method as claimed in claim 1, wherein the handling flow of the analysis result includes comparing the analysis result with a second analysis result.

19. The method as claimed in claim 18, wherein the second analysis result is stored by the user in advance.

20. The method as claimed in claim 1, wherein the handling flows include storing the biosignals and/or the analysis result in the service platform.

21. The method as claimed in claim 20, wherein the user is capable of accessing the stored biosignals and/or analysis result when entering the service platform next time.

22. The method as claimed in claim 1, wherein the handling flows include a medical personnel enters the service platform to access the biosignals and/or the analysis result stored by the user.

23. The method as claimed in claim 22, wherein the service platform plays a communication medium between the user and the medical personnel.

24. The method as claimed in claim 22, wherein the access from the medical personnel is authorized by the user.

25. The method as claimed in claim 1, wherein the handling flows include transmitting the analysis result and/or the biosignals to a medical service provider.

26. The method as claimed in claim 25, wherein the service platform further includes a list of medical personnel who participate in the interactive physiological analysis.

27. The method as claimed in claim 26, wherein the medical service provider is a medical personnel selected by the user from the list.

28. The method as claimed in claim 26, wherein the medical service provider is a medical personnel selected randomly or allocated by the service platform.

29. An interactive real-time analysis method for biosignals, comprising steps of:
providing at least a biosignal acquisition device;
a user connecting the biosignal acquisition device to a service platform through a network;
the user utilizing the biosignal acquisition device to acquire at least one kind of biosignals therefrom;
the biosignal acquisition device transmitting the biosignals to the service platform in real time;
the service platform providing at least a set of processing means for said kind of biosignals with at least an algorithm included therein;
the user selecting at least a desired processing means from the set of processing means;
the service platform performing the selected processing means on the uploaded biosignals for producing an analysis result;
the user deciding a handling flow for the analysis result;
the user deciding a handling flow for the uploaded biosignals; and
the service platform performing the handling flows decided by the user.

30. The method as claimed in claim 29, wherein the biosignal acquisition device is connected to the service platform through a local device, and the acquired biosignals are stored in the local device prior to being transmitted to the service platform.

31. The method as claimed in claim 29, wherein a medical personnel performs a real-time monitor on the biosignal acquisition of the user through accessing the service platform.

32. An interactive real-time analysis method for biosignals, comprising steps of:
providing a service platform;
providing at least a biosignal acquisition device;
a user connecting the biosignal acquisition device to the service platform via a network;
the user utilizing the biosignal acquisition device to acquire at least one kind of biosignals therefrom; and
the biosignal acquisition device transmitting the biosignals to the service platform in real time, p1 wherein
the service platform provides at least a set of processing means for said kind of biosignals with at least an algorithm included therein;
before, during or after the biosignal acquisition, the method further comprises steps of:
the user deciding a handling flow for said acquired biosignals on the service platform; and
the service platform performing the handling flow decided by the user; and
before, during or after the biosignal acquisition, the method further comprises steps of:
the user selecting at least a processing means related to said kind of biosignals from the set of processing means on the service platform;
the service platform performing the selected processing means on the uploaded biosignals for producing an analysis result;
the user deciding at least a handling flow for the analysis result on the service platform; and
the service platform performing the handling flow decided by the user.
